(19) United States
(12) Patent Application Publication
(10) Pub. No.: US 2021/0238110 A1
(43) Pub. Date: Aug. 5, 2021

(54) p-XYLENE PRODUCTION METHOD

(71) Applicant: ENEOS CORPORATION, Tokyo (JP)

(72) Inventors: Yasuhiro ARAKI, Tokyo (JP); Masanari AKIYAMA, Tokyo (JP)

(73) Assignee: ENEOS CORPORATION, Tokyo (JP)

(21) Appl. No.: 17/054,305

(22) PCT Filed: May 13, 2019
(86) PCT No.: PCT/JP2019/018975

§ 371 Date: Nov. 10, 2020

(30) Foreign Application Priority Data
May 15, 2018 (JP) 2018-094054

(51) Int. Cl. C07C 5/41; C07C 15/08; C07C 11/107; C07C 2/24; B01J 27/25; B01J 21/04; B01J 37/02; C07B 63/00

(52) U.S. Cl. CPC C07C 5/41; C07C 15/08; C07C 11/107; C07C 2/24; C07C 2527/25; B01J 21/04; B01J 37/0203; C07B 63/00; C07C 2521/04; B01J 27/25

(57) ABSTRACT

A method for producing p-xylene, comprising: a dimerization step of bringing a first raw material comprising isobutene into contact with a dimerization catalyst comprising at least one selected from the group consisting of Group 9 metal elements and Group 10 metal elements to generate C8 components comprising 2,5-dimethylhexene; and a cyclization step of bringing a second raw material comprising the C8 components into contact with a dehydrogenation catalyst to generate p-xylene by the cyclodehydrogenation reaction of the C8 components.

### TECHNICAL FIELD

The present invention relates to a method for producing p-xylene.

### BACKGROUND ART

p-Xylene is an industrially useful substance as a raw material of terephthalic acid, which is an intermediate raw material of polyester fiber or PET resin. As a method for producing p-xylene, for example, a method for producing p-xylene from raw material containing ethylene (Patent Literature 1) and a method for producing p-xylene from biomass (Patent Literature 2) are known, and various methods for efficiently producing p-xylene have been examined.

### CITATION LIST

### Patent Literature

Patent Literature 1: Japanese Unexamined Patent Publication No. 2011-79815

Patent Literature 2: Japanese Unexamined Patent Publication No. 2015-193647

### SUMMARY OF INVENTION

### Technical Problem

An object of the present invention is to provide a method for producing p-xylene from isobutene as a raw material, wherein the method enables to obtain p-xylene at a high yield.

### Solution to Problem

One aspect of the present invention relates to a method for producing p-xylene, comprising: a dimerization step of bringing a first raw material comprising isobutene into contact with a dimerization catalyst comprising at least one selected from the group consisting of Group 9 metal elements and Group 10 metal elements to generate C8 components comprising 2,5-dimethylhexene; and a cyclization step of bringing a second raw material comprising the above-mentioned C8 components into contact with a dehydrogenation catalyst to generate p-xylene by the cyclodehydrogenation reaction of the above-mentioned C8 components.

In the above-mentioned production method, the use of a specific catalyst in the dimerization step improves the yield of 2,5-dimethylhexene in the C8 components. As compared with C8 components other than 2,5-dimethylhexene (for example, diisobutylene), 2,5-dimethylhexene tends to maintain the reaction activity of the dehydrogenation catalyst in the cyclodehydrogenation reaction over a long period of time. For this reason, p-xylene can be obtained from a raw material containing isobutene at a high yield according to the above-mentioned production method.

A production method according to one aspect may further comprise a separation step of obtaining the above-mentioned first raw material from a petroleum-derived C4 fraction by reactive distillation.

### Advantageous Effects of Invention

According to the present invention, a method for producing p-xylene from isobutene as a raw material, wherein the method enables to obtain p-xylene at a high yield is provided.

### DESCRIPTION OF EMBODIMENTS

Preferred embodiments of the present invention will be described hereinafter. However, the present invention is not limited to the following embodiments in any way.

A method for producing p-xylene according to the present embodiment comprises: a dimerization step of bringing a first raw material comprising isobutene into contact with a dimerization catalyst comprising at least one selected from the group consisting of Group 9 metal elements and Group 10 metal elements to generate C8 components comprising 2,5-dimethylhexene; and a cyclization step of bringing a second raw material comprising the C8 components into contact with a dehydrogenation catalyst to generate p-xylene by the cyclodehydrogenation reaction of the C8 components.

Diisobutylene herein refers to 2,4,4-trimethyl-1-pentene, 2,4,4-trimethyl-2-pentene or a mixture thereof.

In the production method according to the present embodiment, the use of a specific dehydrogenation catalyst in the dimerization step improves the yield of 2,5-dimethylhexene in the C8 components. As compared with C8 components other than 2,5-dimethylhexene (for example, diisobutylene), 2,5-dimethylhexene tends to maintain the reaction activity of the dehydrogenation catalyst over a long period of time. For this reason, p-xylene can be obtained from the raw material containing isobutene at a high yield with the production method according to the present embodiment. It is considered that the same effect as 2,5-dimethylhexene is obtained also with 2,5-dimethylhexadiene having the same carbon skeleton as 2,5-dimethylhexene. For this reason, the C8 components may further contain 2,5-dimethylhexadiene in the production method according to the present embodiment.

The production method according to the present embodiment may further comprise a separation step of obtaining the first raw material containing isobutene from a petroleum-derived C4 fraction.

The steps of the production method according to the present embodiment will be described in detail hereinafter.

(Separation Step)

The separation step is a step of obtaining the first raw material containing isobutene using a petroleum-derived C4 fraction as a raw material.

The C4 fraction herein refers to a fraction having hydrocarbons having 4 carbon atoms as the main components (for example, 80% by mass or more, preferably 95% by mass or more). Examples of the hydrocarbons having 4 carbon atoms include normal butane and isobutane as C4 alkanes, normal butenes (1-butene and 2-butene) and isobutene as C4 alkenes, and butadiene as C4 diene.

It is preferable that the C4 fraction contain C4 alkanes and C4 alkenes. The total contents of the C4 alkanes and the C4 alkenes in the C4 fraction is, for example, 80% by mass or more, and preferably 95% by mass or more.

It is preferable that the C4 fraction contain isobutene from the viewpoint that the first raw material is easily obtained. Since isobutane can be easily converted to isobutene by dehydrogenation, the C4 fraction may contain isobutane. That is, it is preferable that the C4 fraction contain isoforms (isobutane and isobutene) from the viewpoint that the first raw material is efficiently obtained. The content of the isoforms in the C4 fraction may be, for example, 10% by mass or more, preferably 30% by mass or more, and more preferably 40% by mass or more. The upper limit of the content of the isoforms in the C4 fraction is not particularly limited, and may be, for example, 100% by mass or less, may be 95% by mass or less, or may be 90% by mass or less.

Since the C4 fraction is derived from petroleum, the C4 fraction may contain sulfur. The content of sulfur may be, for example, 1000 ppm by mass or less, or may be 10 ppm by mass or less.

For example, a product by the fluidized catalytic cracking of a heavy oil fraction, fractions from crude oil, a product by an ethylene cracker, and the like may be contained in the petroleum-derived C4 fraction.

The heavy oil fraction which is a raw material of fluidized catalytic cracking is not particularly limited, and may be indirect desulfurization light oil obtained from a heavy oil indirect desulfurization device, direct desulfurization heavy oil obtained from a heavy oil direct desulfurization device, an atmospheric residue, deasphalted oil obtained from a heavy oil deasphalting device, or the like.

The catalyst used in fluidized catalytic cracking is not particularly limited, and may be a well-known catalyst for fluidized catalytic cracking. Examples of the catalyst for fluidized catalytic cracking include amorphous silica alumina and zeolite.

In the separation step, for example, isoforms (isobutene and isobutane) are separated from the C4 fraction to obtain the first raw material. Since the first raw material is obtained by separating isoforms from the C4 fraction, the content of the isoforms in the first raw material is higher than the content of the isoforms in the C4 fraction. The separation method is not particularly limited, and examples of the separation method include methods such as reactive distillation, adsorption separation, membrane separation, and a TBA method. As the separation method, reactive distillation is preferable from the viewpoint of economical efficiency. When the rate of the isoforms in the C4 fraction is enough high, separation operation does not need to be performed necessarily, and the C4 fraction can also be used as the first raw material as it is.

Performing the reactive distillation of C4 fraction enables to separate the isoforms (isobutene and isobutane) from normal forms (normal butene and normal butane) while converting 1-butene in the C4 fraction into 2-butene. Converting 1-butene into 2-butene enables to separate the isoforms efficiently by reactive distillation since the boiling point difference from the isoforms increases.

Among the above-mentioned separation methods, the TBA method is a method for hydrating isobutene selectively from the C4 fraction, collecting tertiary butanol (TBA), and dehydrating the obtained TBA to obtain isobutene.

(Dimerization Step)

The dimerization step is a step of using isobutene as a raw material component and bringing the first raw material containing isobutene into contact with the dimerization catalyst containing at least one selected from the group consisting of the Group 9 metal elements and the Group 10 metal elements to obtain C8 components containing 2,5-dimethylhexene. The first raw material may be provided to the dimerization reaction in the form of gas. The Group 9 metal element means a metal element belonging to Group 9 of the periodic table in the long-form periodic table of elements based on the convention of IUPAC (International Union of Pure and Applied Chemistry), and the Group 10 metal element means a metal element belonging to Group 10 of the periodic table in the long-form periodic table of elements based on the convention of TUPAC (International Union of Pure and Applied Chemistry).

In the dimerization step, the first raw material may further contain isobutane as a C4 component other than isobutene. Since isobutane is converted into isobutene by dehydrogenation, isobutane can contribute to the production of p-xylene. Although the first raw material may further contain other C4 components than isobutene and isobutane (isobutane, normal butene, normal butane, and the like), it is desirable that the content of the other C4 components be low from the viewpoints of reaction efficiency and recycling efficiency.

The content of the isoforms (isobutane and isobutene) in the first raw material may be, for example, 80% by mass or more, or is preferably 90% by mass or more, or more preferably 95% by mass or more. The upper limit of the content of the isoforms in the first raw material is not particularly limited, and may be, for example, 100% by mass or less, may be 99% by mass or less, or may be 98% by mass or less.

The first raw material may further contain components other than hydrocarbons. The first raw material may contain, for example, sulfur. The content of sulfur in the first raw material may be, for example, 1000 ppm by mass or less, and it is preferable that it be 10 ppm by mass or less.

In the dimerization step, a dimerization reaction may be performed by bringing raw material gas containing the first raw material into contact with the dimerization catalyst. The raw material gas may contain other components than the first raw material, and may further contain, for example, an inert gas as a diluent. Examples of the inert gas include nitrogen. The raw material gas may further contain other gas such as carbon dioxide and hydrogen.

The isobutene concentration in the raw material gas may be, for example, 10% by mass or more, or may be 50% by mass or more. The upper limit of the isobutene concentration in the raw material gas is not particularly limited, and may be, for example, 100% by mass or less, or may be 90% by mass or less.

The dimerization catalyst may have activity for the dimerization reaction of isobutene and be a catalyst containing at least one selected from the group consisting of the Group 9 metal elements and the Group 10 metal elements. It is preferable that a Group 9 metal element be Co, Ni or Pd, and it is more preferable that the Group 9 metal element be Ni or Pd. The dimerization catalyst may contain one of these components, or may contain two or more. The dimerization catalyst may have, for example, a carrier and a supported metal supported by the carrier. Examples of the carrier include zeolite and mesoporous silica.

In the dimerization catalyst, it is preferable that the total content of the Group 9 metal element and the Group 10 metal element be 1% by mass or more, and it is more preferable that the total content be 2% by mass or more based on the total mass of the dimerization catalyst. It is preferable that the above-mentioned total content be 80% by mass or less, and it is more preferable that the total content be 50% by mass or less based on the total mass of the dimerization catalyst. When the total content of the Group 9 metal element and the Group 10 metal element is in the above-mentioned range, the activity and the selectivity are compatible at a high level.

As an effective method for characterizing the acidity of a solid catalyst, the ammonia TPD (Ammonia Temperature Programmed Desorption) is known widely. For example, C. V. Hidalgo et al., Journal of Catalysis, volume 85, pp. 362-369 (1984) demonstrates that the amount of Bronsted acid sites and the distribution of the acid strengths of Bronsted acid sites can be measured by ammonia TPD.

The ammonia TPD is a method for adsorbing ammonia, which is basic probe molecules, on a sample solid and measuring the amount and the temperature of ammonia desorbed by raising temperature continuously simultaneously. Ammonia adsorbing to weak acid sites desorbs at low temperature (this corresponds to desorption in a low range of adsorption heat), and ammonia adsorbing to strong acid sites desorbs at high temperature (this corresponds to desorption in a high range of adsorption heat). In such ammonia TPD, since the acid strength is indicated with the temperature and the amount of adsorption heat, and a color reaction is not used, the solid acid strength and the solid acid amount are more accurate values. For this reason, examples of a method for evaluating the characteristics of the dimerization catalyst according to the present embodiment appropriately include ammonia TPD.

In the present embodiment, the total acid amount by the ammonia TPD of the dimerization catalyst may be, for example, 1.0 mmol/g or less, and is preferably 0.7 mmol/g or less, and more preferably 0.4 mmol/g or less. The use of a dimerization catalyst having a small acid amount inhibits side reactions in which skeletons other than 2,5-dimethylhexadiene are formed, and enables to obtain 2,5-dimethylhexadiene selectively. The lower limit of the above-mentioned total acid amount is not particularly limited, and may be 0 mmol/g.

In the present embodiment, the total acid amount by the ammonia TPD of the dimerization catalyst indicates a value determined by ammonia temperature programmed desorption (NH₃-TPD) in which the amount of ammonia adsorbed is measured with the device described in “Niwa; Zeolite, 10,175 (1993)” or the like under the conditions described therein.

In the present embodiment, the dimerization catalyst may further contain other metal elements other than the Group 9 metal elements or the Group 10 metal elements. Examples of the other metal elements include Cu, Ag, Au, Fe, Zn, Zr, V and Ti. The dimerization catalyst may not contain the above-mentioned other metal elements.

As the supply source of a metal element supported on the dimerization catalyst, for example, a salt or a complex containing the metal element is used. The salt containing the metal element may be, for example, an inorganic salt, an organic acid salt, or a hydrate thereof. The inorganic salt may be, for example, a sulfate, a nitrate, a chloride, a phosphate, a carbonate or the like. The organic salt may be, for example, an acetate, an oxalate, and the like. The complex containing the metal element may be, for example, an alkoxide complex, an ammine complex or the like.

In the dimerization step, the reaction conditions of dimerization reaction are not particularly limited, and may be optionally changed depending on the activity of a catalyst to be used and the like.

The C8 components containing 2,5-dimethylhexane are generated in the dimerization step. The C8 components are hydrocarbons having 8 carbon atoms, and the hydrocarbons are generated by reacting two molecules of isobutene in the first raw material. The C8 components may further contain, for example, 2,5-dimethylhexadiene. It is preferable that the proportion of 2,5-dimethylhexene and 2,5-dimethylhexadiene in the C8 components be, for example, 50% by mass or more, it is more preferable that the proportion be 70% by mass or more, and it is further preferable that the proportion be 90% by mass or more. When the proportion of 2,5-dimethylhexene and 2,5-dimethylhexadiene is high, in the below-mentioned cyclization step, the selectivity of p-xylene and the reaction activity of the dehydrogenation catalyst tend to be maintained for a long period of time.

In a dimerization step, the first product containing the C8 components is obtained from the first raw material. In the present embodiment, the first product may be used as a raw material of the below-mentioned cyclization step as it is. The first product may further contain, for example, unreacted C4 components (isobutene, isobutane and the like).

Subsequently, the reaction conditions in the dimerization step and the like will be described in detail.

The dimerization step is a step of reacting the first raw material with a dimerization catalyst to obtain 2,5-dimethylhexene.

The dimerization step may be performed, for example, using a reactor filled with the dimerization catalyst by circulating the first raw material in the reactor. Various reactors used for gaseous phase reaction with solid catalysts can be used as the reactor. Examples of the reactor include a fixed bed reactor, a radial flow reactor and a tubular reactor.

The reaction style of the dimerization reaction may be, for example, a fixed bed style, a movable bed style or a fluidized bed style. Among these, the fixed bed style is preferable from the viewpoint of facility cost.

The reaction temperature of dimerization reaction, namely, the temperature in the reactor, may be 50 to 300° C., may be 80 to 250° C., or may be 120 to 200° C., from the viewpoint of reaction efficiency. If the reaction temperature is 50° C. or more, the amount of 2,5-dimethylhexene generated tends to increase further. If the reaction temperature is 300° C. or less, the deterioration is easily suppressed, the side reactions hardly proceed, and high selectivity of the dimerization catalyst therefore tends to be maintained over a longer period of time.

The reaction pressure, namely, atmospheric pressure in the reactor, may be 0.01 to 5 MPa, may be 0.5 to 3.5 MPa, and may be 1.0 to 3.0 MPa. If the reaction pressure is in the above-mentioned range, the dimerization reaction proceeds easily, and still more excellent reaction efficiency tends to be obtained.

When the cyclization step is performed in a continuous reaction style in which the first raw material is fed continuously, the weight hourly space velocity (hereinafter referred to as “WHSV”), for example, may be 1 h⁻¹ or more, or may be 5 h⁻¹ or more. The WHSV may be 1,000 h⁻¹ or less, or may be 100 h⁻¹ or less. Here, the WHSV is the ratio of the speed of raw material gas (the first raw material) fed (fed amount/time) F to the mass of the dimerization catalyst W (F/W). When the WHSV is 1 h⁻¹ or more, the reactor size can be further reduced. When the WHSV is 1,000 h⁻¹ or less, the amount of 2,5-dimethylhexene generated can be further increased. Further preferable ranges of the amounts of the raw material gas and the catalyst used may be selected optionally depending on reaction conditions, the activity of the catalyst and the like, and the WHSV is not limited to the above-mentioned range.

(Cyclization Step)

In a cyclization step, a second raw material containing the C8 components is brought into contact with a dehydrogenation catalyst to obtain p-xylene which is a product of the cyclodehydrogenation reaction of the C8 components. The second raw material may be provided to the cyclodehydrogenation reaction in the form of gas.

The C8 components are hydrocarbons having 8 carbon atoms. It is desirable that the C8 components contain a p-xylene precursor selected from the group consisting of diisobutylene, 2,2,4-trimethylpentane, 2,5-dimethylhexane, 2,5-dimethylhexene and 2,5-dimethylhexadiene, and it is particularly desirable to contain 2,5-dimethylhexene (and 2,5-dimethylhexadiene). It is preferable that the proportion of 2,5-dimethylhexene and 2,5-dimethylhexadiene in C8 components be, for example, 50% by mass or more, it is more preferable that the proportion be 70% by mass or more, and it is further preferable that the proportion be 90% by mass or more.

In the cyclization step, the first product obtained in the dimerization step may be used as the second raw material as it is. That is, the cyclodehydrogenation reaction of the C8 components may be performed in the presence of the C4 components (isobutene and isobutene) contained in the first product in the cyclization step.

In the cyclization step, the cyclodehydrogenation reaction may be performed by bringing the raw material gas containing the second raw material into contact with the dehydrogenation catalyst. The raw material gas may contain other components than the second raw material, and may further contain, for example, an inert gas as a diluent. Examples of the inert gas include nitrogen. The raw material gas may further contain other gas such as carbon dioxide and hydrogen.

The dehydrogenation catalyst may be a catalyst having activity for the cyclodehydrogenation reaction of the C8 components. The dehydrogenation catalyst may have, for example, a carrier and a supported metal supported on the carrier.

As the carrier, an inorganic carrier is preferable, and an inorganic oxide carrier is more preferable. It is preferable that the carrier contain at least one element selected from the group consisting of Al, Mg, Si, Zr, Ti and Ce, and it is more preferable that the carrier contain at least one element selected from the group consisting of Al, Mg and Si. As the carrier, an inorganic oxide carrier containing Al and Mg is used particularly suitably from the viewpoints that side reactions are inhibited, and p-xylene is obtained more efficiently.

Examples of the supported metal include Cr. Pt and Sn. It is preferable that the dehydrogenation catalyst contain at least one supported metal selected from the group consisting of the Group 6 metal elements, the Group 10 metal elements, and the Group 14 metal elements. The Group 6 metal element means a metal element belonging to Group 6 of the periodic table in the long-form periodic table of elements based on the convention of TUPAC (International Union of Pure and Applied Chemistry), and the Group 14 metal element means a metal element belonging to Group 14 of the periodic table in the long-form periodic table of elements based on the convention of IUPAC (International Union of Pure and Applied Chemistry).

One preferred aspect of the dehydrogenation catalyst will be shown below.

The dehydrogenation catalyst of the present aspect is a catalyst in which a supported metal including the Group 14 metal element and Pt is supported on a carrier containing Al and a Group 2 metal element. Here, the Group 2 metal element means a metal element belonging to Group 2 of the periodic table in the long-form periodic table of elements based on the convention of IUPAC (International Union of Pure and Applied Chemistry).

The Group 2 metal element may be at least one selected from the group consisting of, for example, beryllium (Be), magnesium (Mg), calcium (Ca), strontium (Sr) and barium (Ba). Among these, it is preferable that the Group 2 metal element be Mg.

The Group 14 metal element may be at least one selected from the group consisting of, for example, germanium (Ge), tin (Sn), and lead (Pb). Among these, it is preferable that the Group 14 metal element be Sn.

In the dehydrogenation catalyst of the present aspect, the content of Al may be 15% by mass or more, or may be 25% by mass or more, based on the total mass of the dehydrogenation catalyst. The content of Al may be 40% by mass or less.

In the dehydrogenation catalyst of the present aspect, it is preferable that the content of the Group 2 metal element be 10% by mass or more, and it is more preferable that the content be 13% by mass or more, based on the total mass of the dehydrogenation catalyst. It is preferable that the content of the Group 2 metal element be 20% by mass or less, and it is more preferable that the content be 16% by mass or less, based on the total mass of the dehydrogenation catalyst.

In the dehydrogenation catalyst of the present aspect, it is preferable that the content of the Group 14 metal element be 2% by mass or more, and it is more preferable that the content be 4% by mass or more based on the total mass of the dehydrogenation catalyst. It is preferable that the content of the Group 14 metal element be 9% by mass or less, and it is more preferable that the content be 6% by mass or less based on the total mass of the dehydrogenation catalyst.

In the dehydrogenation catalyst of the present aspect, it is preferable that the content of Pt be 0.1% by mass or more, and it is more preferable that the content be 0.5% by mass or more based on the total mass of the dehydrogenation catalyst. It is preferable that the content of Pt be 5% by mass or less, and it is more preferable that the content be 3% by mass or less based on the total mass of the dehydrogenation catalyst. When the content of Pt is 0.1% by mass or more, the amount of platinum per the amount of the catalyst increases, and the reactor size can be reduced. When the content of Pt is 5% by mass or less, Pt particles formed on the catalyst have a suitable size for dehydrogenation reaction, and the platinum surface area per unit platinum weight increases, and thus a more efficient reaction system can therefore be achieved.

In the dehydrogenation catalyst of the present aspect, it is preferable that the molar ratio of the Group 14 metal element to Pt (the number of moles of the Group 14 metal element/the number of moles of Pt) be 3 or more, and it is more preferable that the ratio be 6 or more from the viewpoints that side reaction is inhibited, and the reaction efficiency is further improved. It is preferable that the molar ratio of the Group 14 metal element to Pt be 15 or less, and it is more preferable that the ratio be 13 or less, from the viewpoints that excessive covering of Pt particles by the Group 14 metal element is prevented, and the reaction efficiency is increased.

In the dehydrogenation catalyst of the present aspect, it is preferable that the molar ratio of the Group 2 metal element to Al (the number of moles of the Group 2 metal element/the number of moles of Al) be 0.30 or more, and it is more preferable that the ratio be 0.40 or more, from the viewpoints that side reaction is inhibited and the reaction efficiency is further improved. It is preferable that the molar ratio of the Group 2 metal element to Al be 0.60 or less, and it is more preferable that the ratio be 0.55 or less, from the viewpoint that the dispersibility of Pt in a dehydrogenation catalyst is increased.

The contents of Al the Group 2 metal element, the Group 14 metal element and Pt in the dehydrogenation catalyst can be measured with an inductively coupled plasma-atomic emission spectrometer (ICP-AES) under the following measurement conditions. The dehydrogenation catalyst is subjected to alkali fusion, then converted into an aqueous solution with dilute hydrochloric acid and used for measurement.
Device: manufactured by Hitachi High-Tech Science Corporation, type SPS-3000
High frequency wave output: 1.2 kW
Plasma gas flow rate: 18 L/min
Auxiliary gas flow rate: 0.4 L/min
Nebulizer gas flow rate: 0.4 L/min

The dehydrogenation catalyst of the present aspect has pores having a pore size of 6 nm or more and 18 nm or less (a). The dehydrogenation catalyst may have pores having a pore size of 3 nm or less (hereinafter referred to as “pores (b)”), may have pores having a pore size of more than 3 nm and less than 6 nm (hereinafter referred to as “pores (c)”), and may have pores having a pore size of more than 18 nm (hereinafter referred to as “pores (d)”).

In the dehydrogenation catalyst of the present aspect, the proportion of the pore volume of the pores (a) may be 60% or more of the total pore volume of the dehydrogenation catalyst. When the proportion of the pore volume of the pores (a) is the above-mentioned proportion or more, side reaction is fully inhibited, and sufficient dehydrogenation activity is obtained. It is preferable that the proportion of the pore volume of the pores (a) be 70% or more of the total pore volume of the dehydrogenation catalyst, and it is further preferable that the proportion be 75% or more. The proportion of the pore volume of the pores (a) may be 90% or less of the total pore volume of the dehydrogenation catalyst. The proportion of the pore volumes of predetermined pores can be calculated by analyzing the results measured at a nitrogen relative pressure of 0 to 0.99 by nitrogen adsorption by the BJH method.

It is preferable that the proportion of the pore volume of the pores (b) be 10% or less of the total pore volume of the dehydrogenation catalyst, and it is more preferable that the proportion be 5% or less. The proportion of the pore volume of the pores (b) may be 1% or more of the total pore volume of the dehydrogenation catalyst.

It is preferable that the proportion of the pore volume of the pores (c) be 15% or less of the total pore volume of a dehydrogenation catalyst, and it is more preferable that the proportion be 10% or less. The proportion of the pore volume of the pores (c) may be 5% or more of the total pore volume of the dehydrogenation catalyst.

It is preferable that the proportion of the pore volume of the pores (d) be 30% or less of the total pore volume of a dehydrogenation catalyst, and it is more preferable that the proportion be 20% or less. The proportion of the pore volume of the pores (d) may be 10% or more of the total pore volume of the dehydrogenation catalyst.

It is preferable that the proportion of the total pore volume of the pores (a) and the pores (c) be 70% or more of the total pore volume of the dehydrogenation catalyst, and it is more preferable that the proportion be 80% or more. The proportion of the total pore volume of the pores (a) and the pores (c) may be 95% or less of the total pore volume of the dehydrogenation catalyst.

The specific surface area of the dehydrogenation catalyst of the present aspect may be the same as that of the below-mentioned carrier.

The carrier may be a metal oxide carrier containing, for example, Al and the Group 2 metal element. The metal oxide carrier may be, for example, a carrier containing alumina (Al₂O₃) and an oxide of the Group 2 metal, or may be a composite oxide of Al and the Group 2 metal. The metal oxide carrier may be a carrier containing a composite oxide of Al and a Group 2 metal element and at least one selected from the group consisting of alumina and oxides of Group 2 metal elements. The composite oxide of Al and the Group 2 metal may be, for example, MgAl₂O₄.

The content of Al in the carrier may be 20% by mass or more, or may be 30% by mass or more, based on the total mass of the carrier. The content of Al in the carrier may be 70% by mass or less, or may be 60% by mass or less, based on the total mass of the carrier.

The content of the Group 2 metal element in the carrier may be 10% by mass or more, or may be 15% by mass or more, based on the total mass of the carrier. The content of the Group 2 metal element in the carrier may be 30% by mass or less, or may be 20% by mass or less, based on the total mass of the carrier.

The content of the composite oxide of Al and the Group 2 metal element in the carrier may be 60% by mass or more, or may be 80% by mass or more, based on the total mass of the carrier. The content of the composite oxide of Al and the Group 2 metal element in the carrier may be 100% by mass or less, or may be 90% by mass or less based on the total mass of the carrier.

The content of alumina in the carrier may be 10% by mass or more, or may be 30% by mass or more, based on the total mass of the carrier. The content of alumnina in the carrier may be 90% by mass or less, and may be 80% by mass or less, based on the total mass of the carrier.

The content of the oxide of the Group 2 metal element in the carrier may be 15% by mass or more, or may be 25% by mass or more, based on the total mass of the carrier. The content of the oxide of the Group 2 metal element in the carrier may be 50% by mass or less, or may be 35% by mass or less, based on the total mass of the carrier.

The carrier may contain another metal element besides Al and the Group 2 metal element. The other metal elements may be at least one selected from the group consisting of, for example, Li, Na, K, Zn, Fe, In, Se, Sb, Ni and Ga. The other metal elements may exist as an oxide, or may exist as a composite oxide with at least one selected from the group consisting of Al and the Group 2 metal element.

The carrier may have pores (a), may have pores (b), may have pores (c), and may have pores (d).

The proportions of the pore volumes of the pores (a), the pores (b), the pores (c) and the pores (d) in the carrier may be, for example, similar to the proportions of the pore volumes of respective pores in the above-mentioned dehydrogenation catalyst. A dehydrogenation catalyst wherein the proportions of pore volumes are in the above-mentioned suitable range is easily obtained thereby.

It is preferable that the acidity of the carrier be nearly neutral from the viewpoint that side reaction is inhibited. Here, the standard of the acidity of a carrier is generally distinguished by the pH measured when the carrier is dispersed in water. That is, the acidity of the carrier can be herein indicated by the pH of a suspension in which the carrier is suspended at 1% by mass. The acidity of the carrier may be preferably pH 5.0 to 9.0, and may be more preferably pH 6.0 to 8.0.

The specific surface area of the carrier may be, for example, 50 m²/g or more, and it is preferable that the specific surface area be 80 m²/g or more. The effect of easily increasing the dispersibility of the supported Pt is produced thereby. The specific surface area of the carrier may be, for example, 300 m²/g or less, and it is preferable that the specific surface area be 200 m²/g or less. The carrier having such a specific surface area tends not to have micropores which are easily crushed at the time of firing, in which the carrier is exposed to high temperatures. Therefore, the dispersibility of the supported Pt tends to increase easily. The specific surface area of the carrier is measured with a BET specific surface area meter using nitrogen adsorption.

The method for preparing the carrier is not particularly limited, and may be, for example, a sol-gel method, a coprecipitation method, a hydrothermal synthesis method, an impregnation method, a solid phase synthesis method or the like. The impregnation method is preferable from the viewpoint of facilitating adjusting the proportion of the pore volume of the pores (a) to the above-mentioned suitable proportion.

As an example of the method for preparing the carrier, one aspect of the impregnation method will be shown below. First, a carrier precursor containing a second metal element (for example, Al) is added to a solution in which the precursor of a first metal element (for example, a Group 2 metal element) is dissolved, and the solution is stirred. Then, the solvent is removed at reduced pressure, and the obtained solid is dried. The solid after drying is fired to obtain a carrier containing the first metal element and the second metal element. In this aspect, the content of a target metal element contained in the carrier can be adjusted by the concentration of the target metal element in the solution containing the metal element, the amount of the solution used, and the like.

The metal precursor may be, for example, a salt or a complex containing the metal element. The salt containing the metal element may be, for example, an inorganic salt, an organic acid salt, or a hydrate thereof. The inorganic salt may be, for example, a sulfate, a nitrate, a chloride, a phosphate, a carbonate, or the like. The organic salt may be, for example, an acetate, an oxalate, and the like. The complex containing the metal element may be, for example, an alkoxide complex, an ammine complex, or the like.

Examples of the solvent which dissolves the metal precursor include hydrochloric acid, nitric acid, ammonia water, ethanol, chloroform and acetone.

Examples of the carrier precursor containing the second metal element include alumina (for example, γ-alumina). The carrier precursor can be prepared, for example, by a sol-gel method, a coprecipitation method, a hydrothermal synthesis method, or the like. Commercial alumina may be used as the carrier precursor.

The carrier precursor may have the above-mentioned pores (a). The proportion of the pore volume of the pores (a) in the carrier precursor may be 50% or more of the total pore volume of the carrier precursor, may be 60% or more, or may be 70% or more. In this case, adjusting the proportion of the pore volume of the pores (a) in the dehydrogenation catalyst to the above-mentioned suitable proportion is facilitated. The proportion of the pore volume of the pores (a) may be 90% or less. The proportion of the pore volume of predetermined pores in the carrier precursor is measured in a similar manner as the measurement of the proportion of the pore volume of a predetermined pore size in the dehydrogenation catalyst.

Firing can be performed, for example, in the air atmosphere or an oxygen atmosphere. Firing may be performed in one stage or in multiple stages, which are two or more stages. The firing temperature may be a temperature at which the metal precursor can be decomposed, and may be, for example, 200 to 1000° C., or may be 400 to 800° C. When multistage firing is performed, at least one stage thereof may be performed at the above-mentioned firing temperature. The firing temperature in the other stages may be, for example, in the same range as the above, or may be 100 to 200° C.

As conditions at the time of stirring, for example, the stirring temperature is 0 to 60° C., and the stirring time is 10 minutes to 24 hours. As conditions at the time of drying, for example, the drying temperature is 100 to 250° C., and the drying time is 3 hours to 24 hours.

The supported metal including the Group 14 metal element and Pt is supported on the dehydrogenation catalyst of the present aspect. The supported metal may be supported on the carrier as an oxide, or may be supported on the carrier as a metal which is a simple substance.

Another metal element except the Group 14 metal element and Pt may be supported on the carrier. Examples of the other metal elements are the same as the examples of the other metal element which the above-mentioned carrier can contain. The other metal elements may be supported on the carrier as a metal which is a simple substance, may be supported as an oxide, or may be supported as a composite oxide with at least one selected from the group consisting of the Group 14 metal element and Pt.

The amount of the Group 14 metal element supported on the carrier is preferably 1.5 parts by mass or more, and more preferably 3 parts by mass or more based on 100 parts by mass of the carrier. The amount of the Group 14 metal element supported on the carrier may be 10 parts by mass or less, or may be 8 parts by mass or less based on 100 parts by mass of the carrier. When the amount of the Group 14 metal element is in the above-mentioned range, catalyst deterioration is further suppressed, and high activity tends to be maintained over a longer period of time.

The amount of Pt supported on the carrier is preferably 0.1 parts by mass or more, and more preferably 0.5 parts by mass or more, based on 100 parts by mass of the carrier. The amount of Pt supported on the carrier may be 5 parts by mass or less, or may be 3 parts by mass or less, based on 100 parts by mass of the carrier. In the case of such an amount of Pt, Pt particles formed on the catalyst have a suitable size for dehydrogenation reaction, the platinum surface area per unit platinum weight increases, and a more efficient reaction system can therefore be achieved. In the case of such an amount of Pt, high activity can be maintained over a longer period of time while the catalyst cost is reduced.

The method for supporting the metal on the carrier is not particularly limited, and examples of the method include an impregnation method, a precipitator method, a coprecipitation method, a kneading method, an ion-exchange method and a pore filling method.

One aspect of the method for supporting metal on a carrier will be shown hereinafter. First, a carrier is added to a solution in which a precursor of a target metal (supported metal) is dissolved in a solvent (for example, an alcohol), and the solution is stirred. Then, the solvent is removed at reduced pressure, and the obtained solid is dried. The solid after drying is fired, and the target metal can be supported on the carrier.

In the above-mentioned supporting method, the precursor of the carrier metal may be a salt or a complex containing the metal element. The salt containing the metal element may be, for example, an inorganic salt, an organic acid salt or a hydrate thereof. The inorganic salt may be, for example, a sulfate, a nitrate, a chloride, a phosphate, a carbonate or the like. The organic salt may be, for example, an acetate, an oxalate or the like. The complex containing the metal element may be, for example, an alkoxide complex, an ammine complex or the like.

As conditions at the time of stirring, for example, the stirring temperature is 0 to 60° C., and the stirring time is 10 minutes to 24 hours. As conditions at the time of drying, for example, the drying temperature is 100 to 250° C., and the drying time is 3 hours to 24 hours.

Firing can be performed, for example, in the air atmosphere or an oxygen atmosphere. Firing may be performed in one stage or in multiple stages, which are two or more stages. The firing temperature may be a temperature at which the precursor of the carrier metal can be decomposed, and may be, for example, 200 to 1000° C., or may be 400 to 800° C. When multistage firing is performed, at least one stage thereof may be performed at the above-mentioned firing temperature. The firing temperature in the other stages may be, for example, in the same range as the above, or may be 100 to 200° C.

The degree of dispersion of Pt in the dehydrogenation catalyst of the present aspect may be 10% or more, or may be preferably 15% or more. According to the dehydrogenation catalyst having such a degree of dispersion of Pt, side reaction is further inhibited, and high activity tends to be maintained over a longer period of time. The degree of dispersion of Pt is measured by a method for measuring the degree of dispersion of metal using CO as an adsorption species with the following device and under the following measurement conditions.
Device: Degree of dispersion of metal measuring device R-6011 manufactured by Ohkurariken Co., Ltd.
Gas flow rate: 30 mL/minute (helium, hydrogen)
Amount of Sample: Around 0.1 g (weighed precisely to the fourth decimal place)
Pretreatment: The temperature is raised to 400° C. in a hydrogen air flow over 1 hour, and reduction treatment is performed at 400° C. for 60 minutes. Then, gas is switched from hydrogen to helium, purging is performed at 400° C. for 30 minutes, and cooling is performed to room temperature in a helium air flow. The detector is left to stand at room temperature until the detector becomes stable, and a CO pulse is then performed.
Measurement conditions: First, 0.0929 cm³ of carbon monoxide is pulse-injected every time with helium at normal pressure circulated at room temperature (27° C.), and the amount of adsorption is measured. The number of times of adsorption is performed until the adsorption is saturated (at least 3 times, at most 15 times). The degree of dispersion is calculated from the measured amount of adsorption.

Subsequently, another preferred aspect of the dehydrogenation catalyst will be shown below.

The dehydrogenation catalyst of the present aspect is a catalyst in which a supported metal including Cr is supported on a carrier containing Al.

In the dehydrogenation catalyst of the present aspect, the content of Al may be 40% by mass or more, or may be 50% by mass or more based on the total amount of the dehydrogenation catalyst. The content of Al may be 95% by mass or less.

In the dehydrogenation catalyst of the present aspect, it is preferable that the content of Cr be 5% by mass or more, it is more preferable that the content be 8% by mass or more, and it is further preferable that the content be 12% by mass or more based on the total amount of the dehydrogenation catalyst. It is preferable that the content of Cr be 50% by mass or less, it is more preferable that the content be 40% by mass or less, and it is further preferable that the content be 30% by mass or less based on the total amount of the dehydrogenation catalyst. When the content of Cr is in the above-mentioned range, the yield of p-xylene tends to be improved.

The dehydrogenation catalyst of the present aspect may further contain metals such as Mg, Zr, and K.

When the dehydrogenation catalyst of the present aspect contains Mg, the monomerization of the C8 components to the C4 components is inhibited more remarkably, and p-xylene tends to be obtained more efficiently.

When the dehydrogenation catalyst of the present aspect contains Mg, it is preferable that the content of Mg be 0.1% by mass or more, it is more preferable that the content be 1% by mass or more, and it is further preferable that the content be 1.5% by mass or more based on the total amount of the dehydrogenation catalyst. It is preferable that the content of Mg be 10% by mass or less, it is more preferable that the content be 5% by mass or less, and it is further preferable that the content be 3.5% by mass or less based on the total amount of the dehydrogenation catalyst. When the content of Mg is in the above-mentioned range, the monomerization of the C8 components to the C4 components tends to be inhibited more remarkably.

When the dehydrogenation catalyst of the present aspect contains Zr, side reactions in which skeletons other than p-xylene are formed are inhibited, and the yield of p-xylene in the cyclodehydrogenation reaction tends to be improved.

When the dehydrogenation catalyst of the present aspect contains Zr, it is preferable that the content of Zr be 0.01% by mass or more, it is more preferable that the content be 0.05% by mass or more, and it is further preferable that the content be 0.10% by mass or more based on the total amount of the dehydrogenation catalyst. It is preferable that the content of Zr be 2% by mass or less, it is more preferable that the content be 1% by mass or less, and it is further preferable that the content be 0.50% by mass or less based on the total amount of the dehydrogenation catalyst. When the content of Zr is in the above-mentioned range, side reactions in which skeletons other than p-xylene are formed are inhibited more remarkably, and the yield of p-xylene in the cyclodehydrogenation reaction tends to be further improved.

When the dehydrogenation catalyst of the present aspect contains K, monomerizing to the C4 components and side reactions in which skeletons other than p-xylene are formed are inhibited, and the yield of p-xylene in the cyclodehydrogenation reaction tends to be improved. This effect is more remarkably produced in combination with Zr. That is, the dehydrogenation catalyst of the present aspect may further contain Zr and K.

When the dehydrogenation catalyst of the present aspect contains K, it is preferable that the content of K be 0.1% by mass or more, it is more preferable that the content be 1% by mass or more, and it is further preferable that the content be 1.5% by mass or more based on the total amount of the dehydrogenation catalyst. It is preferable that the content of K be 8% by mass or less, it is more preferable that the content be 5% by mass or less, and it is further preferable that the content be 3% by mass or less based on the total amount of the dehydrogenation catalyst. When the content of K is in the above-mentioned range, monomerization to the C4 components and side reactions in which skeletons other than p-xylene are formed are inhibited more remarkably, and the yield of p-xylene in the cyclodehydrogenation reaction tends to be further improved.

The contents of Al, Cr, Mg, Zr and K in the dehydrogenation catalyst can be measured with an inductively coupled plasma-atomic emission spectrometer (ICP-AES) under the following measurement conditions. The dehydrogenation catalyst is subjected to alkali fusion, then converted into an aqueous solution with dilute hydrochloric acid and used for measurement.
Device: manufactured by Hitachi High-Tech Science Corporation, type SPS-3000
High frequency wave output: 1.2 kW
Plasma gas flow rate: 18 L/min
Auxiliary gas flow rate: 0.4 L/min
Nebulizer gas flow rate: 0.4 L/min

The carrier may be a metal oxide carrier containing, for example, Al. The metal oxide carrier may be, for example, alumina (Al₂O₃), may be a carrier containing alumina (Al₂O₃) and an oxide of a Group 2 metal, or may be a composite oxide of Al and a Group 2 metal. The metal oxide carrier may be a carrier containing a composite oxide of Al and a Group 2 metal element and at least one selected from the group consisting of alumina and oxides of the Group 2 metal elements. γ-Alumina is preferable from the viewpoint that a highly active catalyst having a good affinity for the supported metal is obtained.

The supported metal containing Cr is supported on the dehydrogenation catalyst of the present aspect. The supported metal may be supported on the carrier as an oxide, or may be supported on the carrier as a metal which is a simple substance.

It is preferable that the amount of Cr supported on the carrier be 3 parts by mass or more, it is more preferable that the amount be 5 parts by mass or more, and it is further preferable that the amount be 7.5 parts by mass or more based on 100 parts by mass of the carrier. It is preferable that the amount of Cr supported be 30 parts by mass or less, it is more preferable that the amount be 20 parts by mass or less, and it is further preferable that the amount be 15 parts by mass or less based on 100 parts by mass of the carrier. When the amount of Cr supported is in the above-mentioned range, the yield of p-xylene tends to be improved.

Other metal elements than Cr may be supported on the carrier. Examples of the other metal elements are the same as the examples of the other metal elements which the above-mentioned carrier can contain, and may be Mg, Zr, K and the like. The other metal elements may be supported on the carrier as metals which are simple substances, may be supported as oxides, or may be supported as a composite oxide with Cr.

When Mg is supported on the carrier, it is preferable that the amount of Mg supported be 0.1 parts by mass or more, it is more preferable that the amount be 0.5 parts by mass or more, and it is further preferable that the amount be 1.0 parts by mass or more based on 100 parts by mass of the carrier. It is preferable that the amount of Mg supported be 5 parts by mass or less, it is more preferable that the amount be 4 parts by mass or less, and it is further preferable that the amount be 3 parts by mass or less based on 100 parts by mass of the carrier. When the amount of Mg supported is in the above-mentioned range, the monomerization of the C8 components to the C4 components tends to be inhibited more remarkably.

When Zr is supported on the carrier, it is preferable that the amount of Zr supported be 0.01 parts by mass or more, it is more preferable that the amount be 0.05 parts by mass or more, and it is further preferable that the amount be 0.1 parts by mass or more based on 100 parts by mass of the carrier. It is preferable that the amount of Zr supported be 1 part by mass or less, it is more preferable that the amount be 1.0 parts by mass or less, and it is further preferable that the amount be 0.5 parts by mass or less based on 100 parts by mass of the carrier. When the amount of Zr supported is in the above-mentioned range, side reactions in which skeletons other than p-xylene are formed are inhibited more remarkably, and the yield of p-xylene in the cyclodehydrogenation reaction tends to be further improved.

When K is supported on the carrier, it is preferable that the amount of K supported be 0.1 parts by mass or more, it is more preferable that the amount be 0.5 parts by mass or more, and it is further preferable that the amount be 1.0 part by mass or more based on 100 parts by mass of the carrier. It is preferable that the amount of K supported be 5 parts by mass or less, it is more preferable that the amount be 4 parts by mass or less, and it is further preferable that the amount be 3 parts by mass or less based on 100 parts by mass of the carrier. When the amount of K supported is in the above-mentioned range, the monomerization of the C8 components to the C4 components and side reactions in which skeletons other than p-xylene are formed are inhibited more remarkably, and the yield of p-xylene in the cyclodehydrogenation reaction tends to be further improved.

The method for supporting the metal on the carrier is not particularly limited, and examples of the method include an impregnation method, a precipitator method, a coprecipitation method, a kneading method, an ion-exchange method and a pore filling method.

One aspect of the method for supporting metal on a carrier will be shown hereinafter. First, a solution in which a precursor of a target metal (supported metal) is dissolved in a solvent (for example, water) is prepared. At this time, the amount of water in the solution is adjusted to an equivalent to the pore volume of the carrier. Subsequently, the carrier is impregnated with the solution adjusted to a volume which fills the pores of the carrier. Then, the solvent is removed at low temperature, and the obtained solid is dried. The solid after drying is fired, and the target metal can be supported on the carrier.

In the above-mentioned supporting method, the precursor of the carrier metal may be a salt or a complex containing the metal element. The salt containing the metal element may be, for example, an inorganic salt, an organic acid salt or a hydrate thereof. The inorganic salt may be, for example, a sulfate, a nitrate, a chloride, a phosphate, a carbonate or the like. The organic salt may be, for example, an acetate, an oxalate or the like. The complex containing the metal element may be, for example, an alkoxide complex, an ammine complex or the like.

As conditions at the time of drying, for example, the drying temperature is 100 to 250° C., and the drying time is 3 hours to 24 hours.

Firing can be performed, for example, in the air atmosphere or an oxygen atmosphere. Firing may be performed in one stage or in multiple stages, which are two or more stages. The firing temperature may be, for example, 200 to 1000° C., or may be 400 to 650° C. When multistage firing is performed, at least one stage thereof may be performed at the above-mentioned firing temperature. The firing temperature in the other stages may be, for example, in the same range as the above, or may be 100 to 200° C.

The dehydrogenation catalyst may be a dehydrogenation catalyst other than the above, and for example, a well-known catalyst which can generate p-xylene by the cyclodehydrogenation reaction of the C8 components can be used without particular limitation.

The dehydrogenation catalyst may be molded by a method such as an extrusion method or a tablet compression method.

Unless the physical properties or the catalyst performance of a catalyst are deteriorated, the dehydrogenation catalyst may contain a molding auxiliary from the viewpoint of improving moldability in a molding step. The molding auxiliary may be at least one selected from the group consisting of, for example, a thickener, a surfactant, a water retention agent, a plasticizer, a binder material, and the like. The molding step of molding a dehydrogenation catalyst may be performed in a suitable stage of the production process of the dehydrogenation catalyst in view of the reactivity of the molding auxiliary.

The shape of the molded dehydrogenation catalyst is not particularly limited, and the shape can be suitably selected depending on the form in which the catalyst is used. For example, the shape of the dehydrogenation catalyst may be a shape such as a pellet shape, a granular shape, a honeycomb shape or a sponge shape.

A dehydrogenation catalyst subjected to reduction treatment as a pretreatment may be used. The reduction treatment can be performed by maintaining the dehydrogenation catalyst, for example, in a reducing gas atmosphere at 40 to 600° C. The retention time may be, for example, 0.05 to 24 hours. The reducing gas may be, for example, hydrogen, carbon monoxide or the like.

The use of the dehydrogenation catalyst subjected to reduction treatment enables to shorten an induction period in an early stage of dehydrogenation reaction. The induction period in an early stage of reaction means a state in which the activity of the catalyst is low because, out of the supported metal contained in the catalyst, the supported metal which is reduced to be in an activated state is very little.

Subsequently, reaction conditions in the cyclization step and the like will be described in detail.

The cyclization step is a step of reacting the second raw material with the dehydrogenation catalyst and performing the cyclodehydrogenation reaction of the C8 components to obtain p-xylene.

The cyclization step may be performed, for example, using a reactor filled with the dehydrogenation catalyst by circulating the second raw materials in the reactor. Various reactors used for gaseous phase reaction with solid catalysts can be used as the reactor. Examples of the reactor include a fixed bed reactor, a radial flow reactor and a tubular reactor.

The reaction style of cyclodehydrogenation reaction may be, for example, a fixed bed style, a movable bed style or a fluidized bed style. Among these, the fixed bed style is preferable from the viewpoint of facility cost.

The reaction temperature of cyclodehydrogenation reaction, namely, the temperature in the reactor, may be 300 to 800° C., may be 400 to 700° C., or may be 500 to 650° C., from the viewpoint of reaction efficiency. If the reaction temperature is 300° C. or more, the amount of p-xylene generated tends to increase further. If the reaction temperature is 800° C. or less, the coking speed is not too high, and high activity of the dehydrogenation catalyst therefore tends to be maintained over a longer period of time.

The reaction pressure, namely, the atmospheric pressure in the reactor, may be 0.01 to 1 MPa, may be 0.05 to 0.8 MPa, and may be 0.1 to 0.5 MPa. If the reaction pressure is in the above-mentioned range, dehydrogenation reaction proceeds easily, and still more excellent reaction efficiency tends to be obtained.

When the cyclization step is performed in a continuous reaction style in which the second raw material is fed continuously, the weight hourly space velocity (hereinafter referred to as “WHSV”), for example, may be 0.1 h⁻¹ or more, or may be 0.5 h⁻¹ or more. The WHSV may be 20 h⁻¹ or less, or may be 10 h⁻¹ or less. Here, the WHSV is the ratio of the speed of raw material gas (the second raw material) fed (fed amount/time) F to the mass of the dehydrogenation catalyst W (F/W). When the WHSV is 0.1 h⁻¹ or more, the reactor size can be further reduced. When the WHSV is 20 h⁻¹ or less, the rate of the C8 components converted can be further increased. Further preferable ranges of the amounts of the raw material gas and the catalyst used may be selected optionally depending on reaction conditions, the activity of the catalyst and the like, and the WHSV is not limited to the above-mentioned range.

### EXAMPLES

Hereinafter, the present invention will be described by Examples more specifically; however, the present invention is not limited to Examples.

### Example 1

<Preparation of Catalyst A>

First, 10.0 g of a commercial γ-alumina carrier (manufactured by JGC Catalysts and Chemicals Ltd.) was subjected to impregnation and supporting using an aqueous solution of chromium nitrate (manufactured by Wako Pure Chemical Industries, Ltd., [Cr(NO₃)]6H₃O) so that the amount of Cr supported was 5.0 parts by mass based on 100 parts by mass of the carrier, the resultant was dried at 110° C. overnight, and then fired at 600° C. for 4 hours to obtain a catalyst A.

<Production of p-Xylene>

A C4 fraction obtained by treating Middle East crude oil with a fluidized catalytic cracker was fractionated with a reactive distillation device, isobutane and isobutene were obtained from the overhead, and normal butane and normal butene were obtained from the bottom. The isobutane in overhead gas was 76% by mass, and the isobutene was 24% by mass. This overhead gas was subjected to a dimerization reaction using a fixed bed flow type reactor under the conditions of 200° C., 1.0 MPa, an overhead gas flow rate of 3.3 ml/minute, and a nitrogen flow rate of 16.6 ml/minute to obtain a first product. Then, 1.0 g of Ni 5256 (manufactured by Engelhard Corporation) was used for the catalyst.

In the above-mentioned dimerization reaction, the reaction product 90 minutes after the reaction start was analyzed by a gas chromatograph (HP7890A, manufactured by Agilent Technologies, Inc.). The obtained results are shown in Table 1. In Table 1, the C8 components refer to the total amount of paraffins and olefins generated and having 8 carbon atoms.

Subsequently, cyclodehydrogenation reaction was performed with the fixed bed flow type reactor under the conditions of 500° C., normal pressure and WHSV=1 h⁻¹ using the first product as a raw material. The catalyst A was used for a catalyst. A reaction product from 1 hour ater to 2 hours after the reaction start and a reaction product from 4 hours after to 5 hours ater were collected and analyzed separately. The obtained results are shown in Table 2 as to the rate of 2,5-dimethylhexene converted, the yield of p-xylene, and the fraction of p-xylene in xylene. In Table 2, the yield of p-xylene refers to the yield based on 2,5-dimethylhexene.

TABLE 1

| | |
|---|---|
| 2,5-Dimethylhexene (A) [% by mass] | 0.500 |
| C8 components (B) [% by mass] | 0.545 |
| Ratio A/B | 0.918 |

TABLE 2

Example 1

| | 1 to 2 hours after | 4 to 5 hours after |
|---|---|---|
| Conversion rate | 98.1 | 96.2 |
| p-Xylene fraction | 94.2 | 97.1 |
| p-Xylene yield | 63.0 | 69.3 |

(% by mass)

### Comparative Example 1

<Production of p-Xylene>

A C4 fraction obtained by treating Middle East crude oil with a fluidized catalytic cracker was fractionated with a reactive distillation device, isobutane and isobutene were obtained from the overhead, normal butane and normal butene were obtained from the bottom. The isobutane in overhead gas was 76% by mass, and the isobutene was 24% by mass. This overhead gas was treated with Amberlyst 35, which is a strongly acidic ion-exchange resin, using a fixed bed flow type reactor under the conditions of 120° C., normal pressure and WHSV=50 h⁻¹ to obtain a product of 76% by mass of isobutane, 23% by mass of 2,4,4-trimethylpentene, and 1% by mass of others (a first product).

Subsequently, cyclodehydrogenation reaction was performed with the fixed bed flow type reactor under the conditions of 550° C., normal pressure and WHSV=1 h⁻¹ using the first product as a raw material. The catalyst A was used for a catalyst. A reaction product from 1 hour after to 2 hours after the reaction start and a reaction product from 4 hours after to 5 hours after were collected and analyzed separately. The obtained results are shown in Table 3 as to the rate of 2,4,4-trimethylpentene converted, the yield of p-xylene, and the fraction of p-xylene in xylene. In Table 3, the yield of p-xylene refers to the yield based on 2,4,4-trimethylpentene.

TABLE 3

Comparative Example 1

| | 1 to 2 hours after | 4 to 5 hours after |
|---|---|---|
| Conversion rate | 74.0 | 61.6 |
| p-Xylene fraction | 82.6 | 91.3 |
| p-Xylene yield | 11.7 | 9.3 |

(% by mass)

In the dimerization reaction of Example 1, the proportion of 2,5-dimethylhexene in the C8 components was more than 90 percent. In Example 1, the reaction activity of the dehydrogenation catalyst was maintained over a long period of time, and the rate of 2,5-dimethylhexene converted and the yield of p-xylene were maintained at high proportions as compared with Comparative Example 1.

### INDUSTRIAL APPLICABILITY

According to a method for producing p-xylene according to the present invention, p-xylene can be obtained from C4 components containing isobutene as a raw material at a high yield. p-Xylene is industrially useful as a raw material of terephthalic acid, which is an intermediate raw material of polyester fiber or PET resin.

1. A method for producing p-xylene, comprising:
bringing a first raw material comprising isobutene into contact with a dimerization catalyst comprising at least one selected from the group consisting of Group 9 metal elements and Group 10 metal elements to generate C8 components comprising 2,5-dimethylhexene; and
bringing a second raw material comprising the C8 components into contact with a dehydrogenation catalyst to generate p-xylene by cyclodehydrogenation reaction of the C8 components.

2. The method according to claim 1, further comprising obtaining the first raw material from a petroleum-derived C4 fraction by reactive distillation.
